(19) United States
(12) Patent Application Publication
(10) Pub. No.: US 2011/0118559 A1
(43) Pub. Date: May 19, 2011

(54) NEUROLOGICAL AND/OR PSYCHOLOGICAL TESTER

(76) Inventor: VERED AHARONSON, Ramat Hasharon (IL)

(21) Appl. No.: 13/009,447

(22) Filed: Jan. 19, 2011

Related U.S. Application Data
(62) Division of application No. 11/029,656, filed on Jan. 6, 2005.
(60) Provisional application No. 60/534,387, filed on Jan. 7, 2004.

(51) Int. Cl. A61B 5/00

(52) U.S. Cl. 600/300

(57) ABSTRACT

A method to be performed on a computer for neuropsychological evaluation of a person includes calculating complexities of a plurality of stimuli by a pattern description length algorithm for a pattern of each the stimulus, presenting stimuli to the user in reaction to which the user has to perform a cognitive test, the stimuli being presented in order of complexity and adjusting a complexity level for a next cognitive test based on the user's response.

### CROSS-REFERENCE TO RELATED APPLICATIONS

This application is a divisional application of, and claims priority benefit from, U.S. patent application Ser. No. 11/029,656, filed Jan. 6, 2005, which application claims priority benefit from U.S. Provisional Patent Application No. 60/534,387, filed Jan. 7, 2004, both of which are incorporated herein by reference in their entirety.

### FIELD OF THE INVENTION

The present invention relates to neurological and/or psychological testing generally and to computerization of such in particular.

### BACKGROUND OF THE INVENTION

There have been neuropsychological tests for many years. Such tests diagnose neurological and mental disorders and diseases. Specifically, neuropsychological tests are used for the diagnosis of dementia and geriatric mental diseases. Typically, these tests are manually administered and taken. However, the article, “Human-Computer Interaction in the Administration and Analysis of Neuropsychological Tests,” by Vered Aharonson and Amos D. Korczyn, Computer Methods and Programs in Biomedicine (2004), Vol. 73, pp. 43-53, discusses a computerized neuropsychological assessment unit, described in FIG. 1, to which reference is now made.

The unit, labeled 10, includes a computer 12, a tester 14 and an analyzer 16. Tester 10 provides standard neuropsychological diagnosis tasks on a monitor 18 and/or speakers 19 of computer 12. Analyzer 16 measures a subject's presses on a keyboard 20 in response to the tasks. Analyzer 16 determines reaction parameters from the key press data and changes the tasks and instructions in response to the subject's parameters, regulating the complexity as a function of how well the subject responds. Moreover, analyzer 16 analyzes the reaction time data after the subject has finished the tasks to provide performance analysis of the tests.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram illustration of a prior art computerized neuropsychological assessment unit;

FIG. 2 is a block diagram illustration of a neuropsychological testing system, constructed and operative in accordance with the present invention;

FIG. 3 is a block diagram illustration of a test editor 26 forming part of the system of FIG. 2;

FIG. 4 is a block diagram illustration of an exemplary testing unit, forming part of the system of FIG. 2;

FIG. 5 is a flow chart illustration of the operations of an exemplary testing unit, forming part of the system of FIG. 2;

FIGS. 6A, 6B and 6C are schematic illustrations of a cursor movement analysis, useful in understanding the operation of an analyzer forming part of the system of FIG. 2; and

FIG. 7 is a schematic illustration of a simplified keyboard and display, useful in understanding keyboard spatial analysis performed by the analyzer of FIG. 2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that the basic paradigm of neurological/psychological tests is very uniform and is as follows: Each test battery consists of a sequence of tests. The sequence and the number of the tests may either be pre-defined by the researcher or dynamically modified during the test flow depending on user's reactions.

Each test may consist of 3 main parts:

1) an explanation;

2) training in the test; and

3) the subtests themselves.

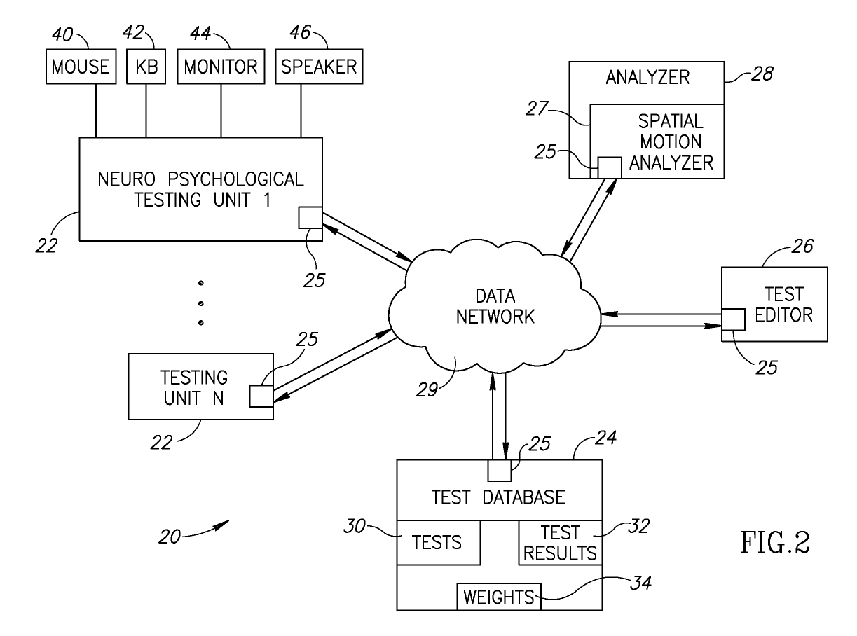

Reference is now made to FIG. 2, which illustrates a neuropsycho logical testing system 20 which may separate the test design from the execution and/or analysis of the tests. Testing system 20 may comprise a test editor 26 in which to generate the tests, a multiplicity of testing units 22 to run the tests, a test database 24 to store the tests and results, and an analyzer 28 to analyze the results. Because the operations are separate, they may be physically present in separate locations, communicating through a data network 29, such as a local area network, an intranet, or the Internet. In one embodiment, each unit 22, 24, 26 and 28 may comprise a communication unit 25, such as one written in the Java language, through which data may pass from one unit to the next.

Test editor 26 may provide an environment in which to prepare test scripts, such as test scripts 30 stored in test database 24. Each test script may describe a test or series of tests to be performed at one sitting. Each test may comprise a set of explanations, a practice test and the subtests. Each subtest may comprise a set of stimuli (visual or aural), preferably from standardized neuropsychological tests, and a set of questions or actions to be asked of the subject with respect to the stimuli. Included in the subtest definition may be the expected answers and the expected timing of the answers.

The test designer may design explanations in any suitable manner. For example, they may be written explanations to be displayed and/or they may be voiced. The latter may be provided through a recording of someone reading the text or through a text-to-speech device (not shown), such as is commonly known.

The test designer may design the practice test series and may define the passing grade, if necessary, to move to the ‘real’ tests. The test designer may define the type of stimuli and the location in test database 24 where the stimuli may be found. For example, some stimuli may be images. Others might be recordings. The test designer may also associate complexity levels with the stimuli and may have multiple complexity levels for a given test. The complexity levels may be based on the complexity levels in the standardized, manual tests or may be defined by the test designer.

The test designer may also define the expected response to each stimulus. These responses may be key presses, cursor movements and cursor clicks. The expected response may also include the expected timing of the response. For example, the expected response of ‘L’ may be required to be received within 0.25 sec. For cursor movements, the expected response may be defined by an optimal trajectory from the starting location to the final location and by the speed and/or direction at which the cursor may be moved. The test may require cursor clicks to occur within a period of time after the cursor arrives at the location. The test may require that the motion be finished within a predefined length of time.

Attached to each testing unit 22 may be a mouse or other cursor unit 40, a standard or customized keyboard 42, a monitor 44 and a speaker 46. Each testing unit 22 may download a selected test script 30 from test database 24 and may then run test script 30. When running test script 30, testing unit 22 may provide the stimuli listed in test script 30 to a subject and may collect his/her responses. Typical response data may include key presses and cursor movements. They may also include timing of when such occurred with respect to given stimuli.

Testing unit 22 may analyze some of the subject's responses to determine if it is possible to move to more complex stimuli and/or to modify the next expected reaction time. In addition, testing unit 22 may provide the full set of responses as test results 32, typically through data network 29, to database 24.

Analyzer 28 may retrieve tests results 32, through data network 29, and may analyze them at any appropriate time. The analysis may occur at predetermined times after the test has finished, at regular intervals or at any other suitable moment. Analyzer 28 may perform the analysis discussed in the article by Aharonson and Korczyn, discussed hereinabove. Alternatively or in addition, analyzer 28 may perform spatial motion analysis with a spatial motion analyzer 27, operative to analysis the motion of a cursor (such as mouse) and/or the motion of the hands over the keys of a keyboard.

Minimally, analyzer 28 may determine a set of features fᵢ from test results 32 and may determine a score S for each subject. The set of features fᵢ may be those discussed in the article by Aharonson and Korczyn and/or may include cursor movement features fᵢ determined by cursor movement analyzer 27. Score S may be determined by:

$$S = {\sum }_{i}^{ } {w}_{i} {f}_{i} < T$$

where T is a threshold defining a disease and wᵢ are empirically determined, per feature weights. The weights wᵢ may be determined for a given population. In one embodiment, the weights were derived from the data of an initial experiment and a follow-up experiment. Through a boost search algorithm, the weights that match best the subjects' cognitive decline towards disease or disorder were calculated. In an alternative embodiment, the weights may be dynamically refined.

The weights may be preferably stored as weights 34 in database 24. Different populations may have different sets of weights 34 and analyzer 28 may select the appropriate set of weights 34 for the subject when performing the analysis.

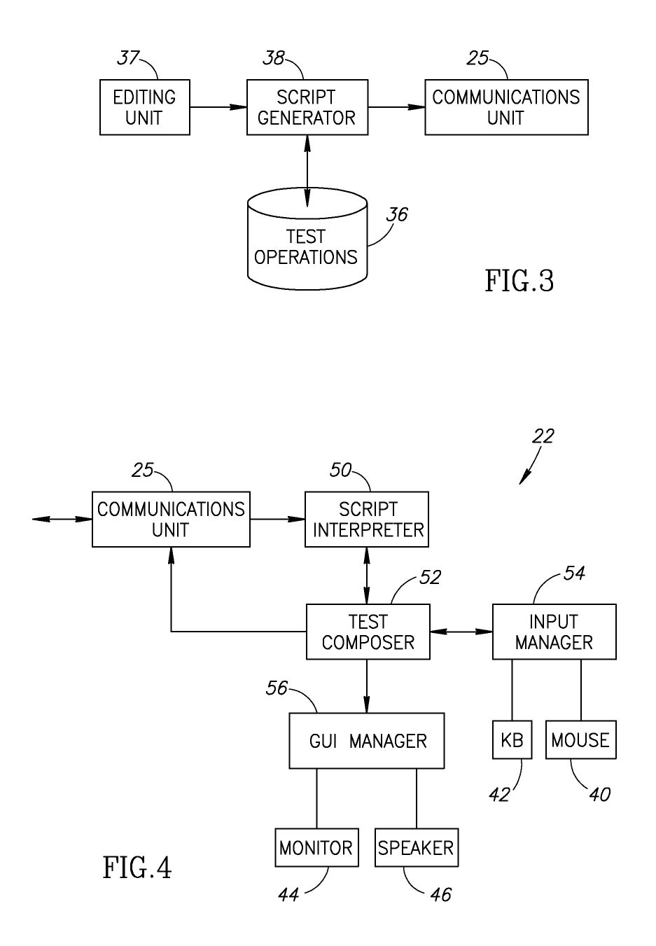

Reference is now made to FIG. 3, which illustrates an exemplary test editor 26. Test editor 26 may comprise a test operation storage unit 36, an editing unit 37, a script generator 38 and one of communication units 25. Within editing unit 37, a test designer may define test batteries, tests, SRPs (stimulus-response pairs, the minimal unit of user/system interaction), test results and subject information.

Each SRP may comprise 2 parts: computer stimuli and their expected user response. Each subtest may be a sequence of SRPs and the stimuli may be sentences of instructions, sentences of explanation, sentences of comments, a visual pattern/symbol/picture, and/or sound or speech.

The test designer may define the amount of stimuli, their types, the desired response for each stimulus and the maximal response time to be allowed. For each stimulus, the test designer may define the stimulus type, the associated audio file, any associated bitmap(s) or a rule (description) for creating the bitmap(s) on the fly, its location on the screen and any rule(s) for selecting the next SRP. For example, the selection rules might be: a random selection, a selection adaptive to user reactions, selections in ascending/descending complexity level, etc. Finally, the test designer may define a format for the results. For example, the test results may be stored as raw data or as summaries.

Test operation storage unit 36 may store code associated with the various types of operations that a test designer may select within editing unit 37.

Script generator 38 may convert the test designer's selections into a test script 30. In the exemplary embodiment, test scripts 30 are XML documents written using an XML Schema. Alternatively, they can be any other suitable document which may be read by testing units 22.

Generator 38 may access storage unit 36 for the code associated with each selection of the test designer. Generator 38 may also add any additional code to generally define the operations to be done.

Reference is now made to FIG. 4, which illustrates an exemplary testing unit 22 which may operate with test scripts written in XML and software written using the Java language. Other forms of operation are possible and are included in the present invention.

Each unit 22 may comprise its communication unit 25, a script interpreter 50, a test composer 52, an input manager 54 and a graphical user interface (GUI) manager 56. Input manager 54 may connect to the input units, such as keyboard 42 and mouse 40. GUI manager 56 may control monitor 44 and speaker 46.

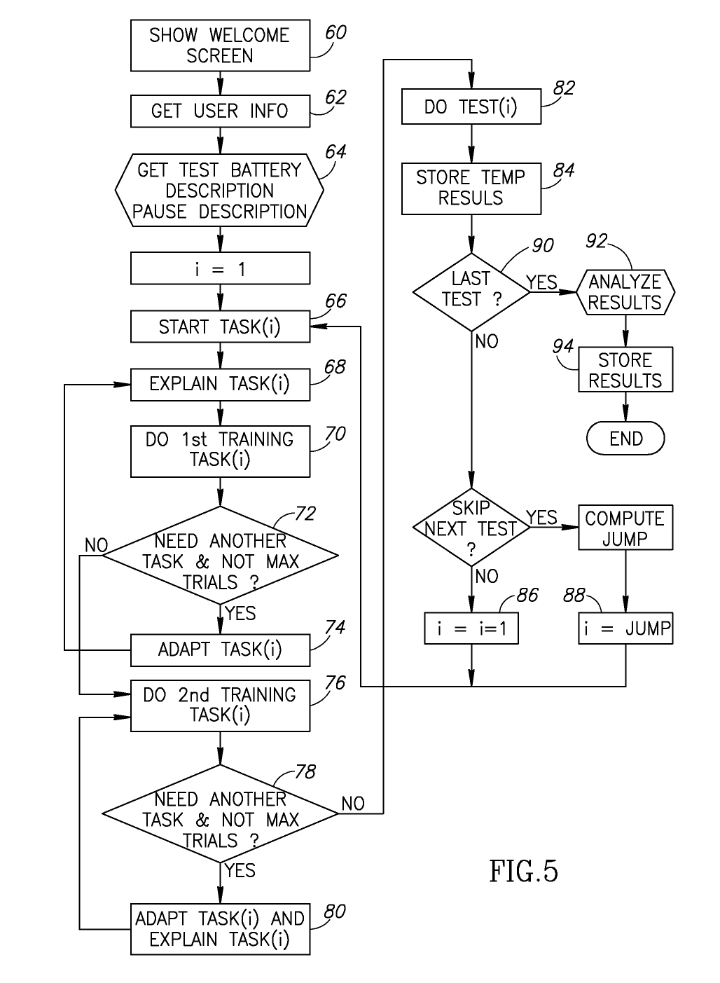

Test composer 52 may run a selected test. To do so, it may first call communication unit 25 to retrieve the specified test script 30 from database 24. Composer 52 may call script interpreter 50 to convert the retrieved test script 30 to a set of Java classes and may then build and run the test with the Java classes. The running of a test is described in more detail hereinbelow, with respect to FIG. 5.

Composer 52 may store the subject's responses during the test battery and may call script interpreter 50 to convert the test results to XML. Finally composer 52 may call communication unit 25 to store the test results in database 24.

Communication unit 25 may be written in Java and may connect each test unit 22 and database 24. It may handle all communication and/or network operations. In addition, it may handle database operations, such as GET and PUT operations, and converting requests from test composer 52 into standard database requests, such as SQL queries. It may also receive query results from database 24 and may pass the results to the request originator.

Script interpreter 50 may convert between test scripts 30, (in this example, written in XML), and a set of programming language classes (in this example, Java). For converting from XML, interpreter 50 may get references to an empty set of Java classes, may run a standard XML parser to convert XML data to Java classes and may return the Java classes to the calling routine For converting to XML, interpreter 50 may get references to a filled set of Java classes, may walk through the classes, extracting data and converting them back to an XML file and may return the XML file to the caller.

Reference is now made to FIG. 5, which illustrates an exemplary operation for running a test battery. For each test in the test battery, there are two training sessions followed by the actual test. The first training session typically may be relatively simple while the second training session may be a more complex version of the same type of task. The actual test may provide multiple tasks of the same type, some simple and others complex.

In step 60, composer 52 may show a welcome screen after which (step 62), composer 52 may get the subject information, typically according to a dialog screen. In step 64, composer 52 may request test script 30 from database 24 (through communication unit 25) and may request that script interpreter 50 convert it. After this set up, composer 52 may run the test.

The test may comprise multiple tasks, which composer 52 may run sequentially in the loop of steps 66-88. For each task, composer 52 may first initiate the task (step 66). In step 68, composer 52 may provide the test explanation, as indicated in test script 30. In step 70, composer 52 may run the first training task, displaying the stimuli defined for it and receiving the subject's responses. If the subject requires another trial (as checked in step 72), composer 52 may review the data and may make (step 74) the task more or less complex to adapt to the subject's responses. Composer 52 may repeat the process (from step 68) until the subject either has mastered the task (according to the definitions in test script 30) or has achieved the maximum number of trials (as listed in test script 30). The check is performed in step 72.

Composer 52 may continue (step 76) with a second training session, using the stimuli defined for it. If the subject requires another trial (as checked in step 78), composer 52 may review the data and may make (step 80) the task more or less complex to adapt to the subject's responses. Composer 52 may repeat the process (from step 76) until the subject either has mastered the task (according to the definitions in test script 30) or has achieved the maximum number of trials (as listed in test script 30). The check is performed in step 78.

Finally, in step 82, composer 52 may provide the test for which the subject has been trained. In this step, composer 52 may take the data and may analyze it to determine when to make the tasks listed therein more complex. Such an analysis is discussed in the above-mentioned article by Aharonson and Korczyn.

After running the test, composer 52 may store the data (step 84), and set up to do the next test, which may either be the next one listed (step 86) or another one later on in test script 30 (step 88). If the test battery has finished, as checked in step 90, composer 52 may analyze and store the results (steps 92 and 94).

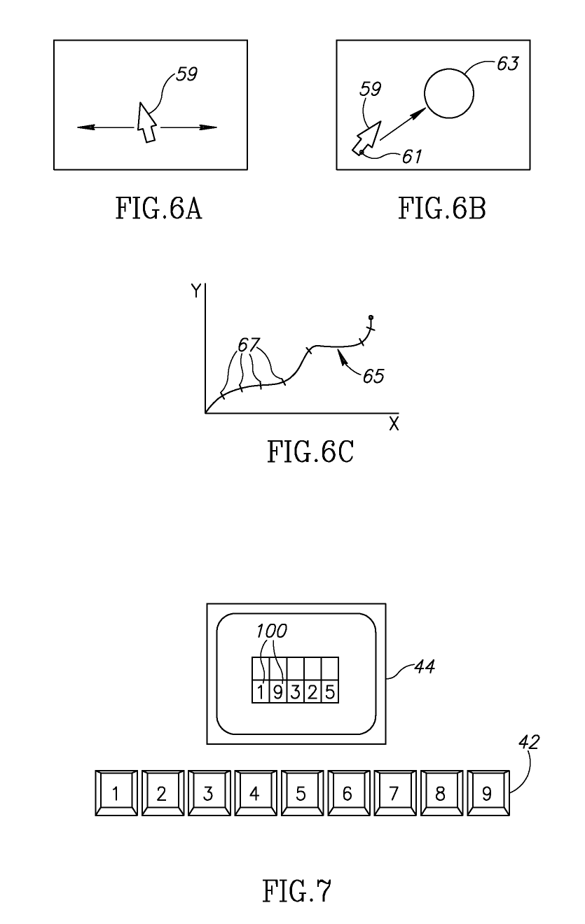

Reference is now made to FIGS. 6A, 6B and 6C, which illustrate aspects of the cursor movement analysis of analyzer 27. FIGS. 6A and 6B illustrate two types of cursor movement tests. In the test of FIG. 6A, the subject may be told to move a cursor 59 back and forth and in the test of FIG. 6B, the subject may be told to move cursor 59 from a starting point 61 to a button 63 and to select button 63, such as by clicking on it. Testing unit 22 may record the cursor trajectories.

Spatial motion analyzer 27 may determine features related to the quality of cursor movement. To do so, analyzer 27 may divide each cursor trajectory, shown in FIG. 6C as a curve 65, into a multiplicity of linear segments 67. For each segment, analyzer 27 may determine the speed, a feature v1, and the variance of the movement from a straight line. The latter may be a feature v2. Analyzer 27 may then average the values of features v1 and v2 over the line segments 67. Analyzer 27 may determine the jerkiness of the subject's motion as a function of how many segments the trajectory must be divided into.

For the movement of the type of FIG. 6B, spatial motion analyzer 27 may determine the subject's manner of stopping cursor 59 at button 63 (whether in a stable manner or with much stopping) and the location of the stop (a feature v4) with respect to the center of button 63. The stopping manner may be determined by counting the number of crossings in and out of button 63, a feature v3.

Spatial motion analyzer 27 may also determine the click latency (a feature v5) as a measure of how long after the subject brought cursor 59 to button 63 did s/he click button 63. Finally, analyzer 27 may determine click persistency (a feature v6) as a measure of how long the subject pushes on button 63 (i.e. from click on to click off).

Reference is now made to FIG. 7, which is useful in understanding the operation of spatial motion analyzer 27 when analyzing key presses. Testing unit 22 may display an image, on monitor 44, of some numbers 100 for the subject to type using keyboard 42. FIG. 7 shows only the number keys of keyboard 42. As can be seen from FIG. 7, some of the keys, such as the 1 and the 2 keys, are close to each other while other keys, such as the 1 and the 9 key, are further apart.

Applicant has realized that, due to the spatial relationship of the keys, it will take longer to press keys that are apart from each other than those which are nearby. Thus, analyzer 27 may normalize the reaction time data of subsequent key presses as a function of the spatial relationships of the keys to each other. The spatial relationship may be expressed in absolute or relative distance between the keys.

For example, for the keys 100 indicated on monitor 44 of FIG. 7 (i.e. 1, 9, 3, 2 and 5), the reaction times may be normalized so that the relationship of each key to its subsequent key is: 8, 6, 1 and 3, which defines the number of keys on keyboard 42 between subsequent key presses.

### Appendix A

### I. Detailed Description of the Tests Presentation

All tests employ a similar format of presentation. The features of this format were developed after testing a group of 5 pre-control subjects daily, when every change in features, was evaluated by the pre-controls till the final format of the software established.

### Oral Instructions Interface

The instructions and explanations and comments during the trial period are oral. Since the tests themselves are not language- or culture-dependent, it suffice to translate and record the instruction sentences to a different language, and the battery can be readily used in any country.

### Presentation Screens Colors

The tests are governed by on the interaction of subjects with the computer through the graphical user interface. The features of this GUI have thus been carefully established. The background screen color is soft blue. The test stimuli are presented inside an inner frame, with a more horizontal aspect ratio (16:9 instead of the 4:3 for the current computer screens). This approximates more closely the aspect ratio of the human vision and enhances peripheral vision, to the left and right of visual focus. The test objects are presented in dark blue. This color combination has a pleasant effect as well as maximizes the contrast without having a sustained visual effect, (like black on light background).

### Trial Interface

The instructions are followed by a trail period. The trial period's examples and comments are oral and the trials are adapted to the subject's performance. Throughout the trial period, each movement of the subject is recorded and each stage of the trial tallies with the previous stage's functioning. Based on the subject's performance the oral explanations are recapitulated and rephrased for several times, until full understanding of the test instructions is achieved. The instructions are abbreviated if the trial performance of the subject is skillful. In each trial period, a limit number of trials is defined. When this limit is reached, the subject is thanked and released without having to perform the test.

### Pre-Test Interface

The trial period is followed by the sentence “press a key when you're ready to start”. If the subject does not press a key, the sentence is repeated. After three repetitions, the test is aborted.

### Test Stimuli

### Complexity Calculation and Adaptation Mechanism

The tests contain presentation of stimuli, either visual or auditory, in reaction to which the subject has to perform a cognitive task. The stimuli are random and presented in an order of complexity, adaptive to the subject's performance: Upon a correct performance of the subject for a certain stimulus, the next stimulus' complexity will be increased, and Upon an incorrect performance, the next stimulus' complexity will be decreased. The complexity of each stimulus is calculated using the Pattern Description Length (PDL) algorithm:

### Complexity Algorithm Description

Most image complexity measures strongly depend on the method of the image tracing (i.e. row-by-row, column-by-column). Tracing is the process of searching through text to find other references to the same term or symbol [Cant95].

Each tracing of a pattern produces a different code. The challenge is to define a measure for both this code's complexity.

We first define the main screening method of the 4*4 squares matrix, used in our visual identification and recall tasks:

The tracing can be started either from the four corner squares of the matrix (indices [1,1], [1,4], [4,1] and [4,4]) or from the four inner squares (indices [2,2], [2,3], [3,2] and [3,3]).

Starting from a corner square, one can trace to either left or right, in three main modes: 1. row after row or column after column, all in the same direction. 2. changing direction after each row or column. 3. circle inward.

Starting from an inner square, one can trace to either left or right, in circular motion outward. There are therefore 4*2*3+4*2=32 tracing modes.

One can, however, use symmetry to discard some of modes, regarding the tracing paths as similar: circular tracing to left or to right in this matrix is symmetrical. Similarly, the 4 corners are symmetrically interchangeable, and so are the 4 inner squares. One is, thus, left with 1*5+1*1=6 distinct tracing modes.
1. row-by-row, starting from a corner square, from left to right, or right to left.
2. row-by-row, starting from a corner square and changing tracing direction in each row.
3. column-by-column, starting from a corner square, from top to bottom or bottom to top.
4. column-by-column, starting from a corner square and changing tracing direction in each column.
5. inward spiral, starting from any corner of the matrix (i.e. start from leftmost top square, trace right till rightmost, then down to rightmost bottom, then left to rightmost bottom and up till second from top square. Continue this tracing in a decreasing square path, till the inner 4 squares of the matrix).
6. outward spiral, starting from any of the matrix's inner 4 squares and tracing outward.

Each tracing method yields a sequence of 16 elements. Writing “1′” for each black square and “0” for each white one, one has a word of 16 bits, Wᵢ, i=1:6, for every tracing for each pattern.

A word description length, n, was therefore defined as

n=L−S (1)

where L is the Lempel-Ziv code length and S the description-facilitating sequences measure.

The pattern description length (PDL) algorithm is as follows:

```
    For a given pattern j
    For each tracing i, i=1:6
    {
    Calculate nⱼ(i), the number of bits required to describe the
word created from pattern j using tracing i.
    }
```

Calculate p(i), the a-priori probability for compression using each tracing i.

Calculate the complexity of word j in bits:

PDL(j)=minᵢ[nⱼ(i)+log pⱼ(i)]. (2)

The minimization argument of the PDL includes two terms. If the use of a less likely tracing yielded a shorter description length, this should be taken into consideration, therefore [nⱼ(i)+log pⱼ(i)] includes both the compression length of each word j, and the a priori term which describes the penalty for using an unlikely tracing method i, that created this word from the pattern. The minimization over all tracing methods of this term thus gives the minimal number of bits required to describe the pattern j.

The challenge in this reasoning is to choose the appropriate a priori probability. A “mathematical” choice will be to calculate the PDLs for a large population of patterns and to count and normalize the number of times each tracing methods yielded a minimum. It is very unlikely, however, that the human visual perception can grasp and handle such calculations. However, many research of vision and eye movement (i.e.Yarbus, 1967; Rayner, 1992) imply that objects detection starts in the center of the figure/pattern. Based on this assumption, the a priori probability should favour the sixth tracing method—starting from the middle of the pattern. Working a-priori probabilities were therefore chosen: 0.15, 0.15, 0.15, 0.15, 0.15 and 0.25 for the first through the sixth tracing methods.

The complexity values were calculated for all the 2¹⁶ patterns and then normalized to an index P_{C}, between 0 and 1.

In most perceptual tests, the subjects are not presented with a single stimulus, but with two or more, and the tasks entail comparison between the stimuli. Determining task complexity, thus, necessitates a measure reflecting the ratio between the complexities of the patterns. A complexity ratio R_{PC} was defined between any two different patterns, A and B, such that the “simpler” pattern will always be in the denominator. Therefore, for each different patterns pair, A and B that fulfils P_{C}(A)<P_{C}(B), the task complexity index is:

R_{PC}=P_{C}(A)*P_{C}(A)/P_{C}(B)

R_{PC} thus decreases when the difference between the patterns is larger and the task is easier and increases proportionally to the complexity of the less difficult pattern.

### Tests Order

The order of the test presentation is determined by the complexity of computer interface usage. The first test involves pressing any key on the keyboard. The second and third tests involve choosing and pressing one of three specific keys (the digits “1”, “2” or “3”). The fourth test involves choosing and pressing one of nine specific keys (the digits “1”, “2” till “9”). The fifth and sixth tests involve choosing and pressing one of ten specific keys (all the digits from “0” till “9”). The seventh test involves using the mouse.

### Individual Tests GUI Details

### Spot the Plus Symbols

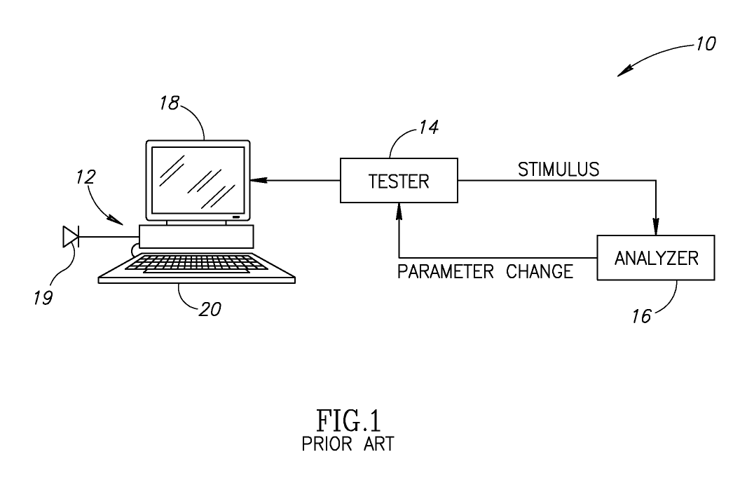

In this part, the subject is presented with symbols that appear in the center of the screen. The height of a symbol is 0.1 of the screen's height and the width of a symbol is 0.1 of the screen's width (FIG. 1). Each symbol appears for 1.5 sec and the interval between symbols' appearance is 1.5 sec. A sequence of 20 symbols is presented. The probability for “+” appearance is 0.5. Six other symbols are presented in “&”, “′”, “$”, “#”, “!” and “%”, each with a probability of 0.5/6. The subject is instructed to press on any key on the keyboard, when he/she sees a Plus (“+”) symbol on the screen.

### Find the Different Pattern

In this part, the subject is presented with three patterns. A pattern consists of 4 rows of 4 squares each, in which each square can randomly take a white or a dark blue color. The 4×4 squares patterns are centered on the screen and each has a height and width of 4/27 the screen width. Beneath each pattern is a number: 1, 2 and 3. In each trial two different patterns are drawn—one applied to two of the patterns on the screen and the other to the third one. Thus two of the patterns (distractors) are always similar and one is different. The filling of the squares that consist of a pattern is random but is governed by two complexity indices that are determined for every trial, according to the subject's performance in the previous trial. The visual complexity of the task R_{PC} was calculated using the P_{C} of the odd pattern and of the two identical distractors. The initial index is Rpc=0.5. If the subject correctly differentiates between the patterns, the next Rpc is changed according to the following rule:

Rpcₙₑₓₜ=Rpc+0.125*Rpc where if Rpcₙₑₓₜ>0.999, the test terminates.

If the subject does not choose correctly the different pattern, the next (K1, dK) are changed according to:

Rpcₙₑₓₜ=Rpc−0.25*Rpc and where if Rpcₙₑₓₜ<0.333, the test terminates.

The subject is instructed, to press the keyboard key of number that shows under the pattern that is different. Each screen stays on until the subject presses a digit key—1, 2 or 3. When the subject presses a key, the corresponding number is highlighted on the screen, indicating the chosen pattern and the next screen is presented. No other key pressing, beside the digits from 1 to 3, is accepted (highlighted).

The total length of the test is 100 seconds or less, in case of good performance, or a 25 seconds presentation of the first test screen, in case that the subject has not responded at all.

### Recall of a Pattern

In this part, the task consists of a pair of two screens. In the first screen, the subject is presented with a single pattern and is instructed to remember it. A pattern consists of 4 rows of 4 squares and is implemented like the patterns of test 2. The pattern then disappears and after 5 seconds three patterns appear, in a similar screen order as in test 2. One of the patterns is similar to the single pattern that disappeared and the other two patterns (distractors) are different. Like in test 2, the patterns contents are random but governed by two complexity indices that are determined for every trial, according to the subject's performance in the previous trial. Here, the visual complexity of the task, R_{PC} calculation used mean P_{C} of the two distractors and that of the to-be-recalled pattern. The initial indices and their increments are similar to the ones defined for test 2.

In the second screen beneath each pattern is a number: 1, 2 and 3. The subject is instructed to press the keyboard key of number that shows under the pattern that is similar to the single one that appeared on the last screen. Each 3-patterns screen stays on until the subject presses a digit key—1, 2 or 3. When the subject presses a key, the corresponding number is highlighted on the screen, indicating the chosen pattern and the next screen is presented. No other key pressing, beside the digits from 1 to 3, is accepted (highlighted).

The total length of the test is 250 seconds or less, in case of good performance, or a 40 seconds presentation of the first test screen-pair, in case that the subject has not responded at all.

### Digit-Symbol Substitution Test (DSST)

In this part, a template of is presented in the upper part of the screen. The template consists of two rows of squares. In the first row each square contains a symbol, built of 1-3 line segments. In the second row, each square contains a digit, from 1 to 9. The symbols are similar to the 9 symbols matching the digits in a written DSST test. The subject is instructed that each symbol can be substituted with the digit below it. Below the template a similar table appears, in which the symbols line is filled with randomly chosen symbols and the digits line is empty. During the task, symbols are highlighted, one at a time, together with the empty square below it. The subject is instructed to type (substitute) the digit corresponding to the symbol highlighted, according to the template above. The digit typed is written in the empty square and the next symbol-empty square pair is highlighted. No other key pressing, beside the digits from 1 to 9, is accepted (written in the square). When a table is completely filled by the subject, another screen appears, with the template above and another table to fill below it. The task entails filling as many symbols as possible during 90 sec.

The symbols for the filling tables are randomly chosen, but in every table two adjacent symbols are set to be similar. The filling pattern of such two adjacent symbols was found to be an important feature of the subjects' performance.

### Recall of a Digits Sequence

In this part a sequence of digits is read, more digits every time (i.e. a sequence of 1, 2, 3 . . . till 6 digits). After each sequence read a white square appears on the screen and the subject is instructed to type the digit/digits read on the keyboard. The digits typed are printed in the white square and the next sequence is read. No other key pressing, beside the ten digits from 0 to 9, is accepted (written in the square). If the number of digits typed is smaller that the number of digits read, the white square is sustained for a time delay that equals to three times the longest delay between two subsequent presses of the subject in the present sequence trial, or if no two subsequent presses were recorded, the longest delay is taken from the last sequence trial.

The complexity of the task is determined by the length of the sequence read (from one digit to 6 digits sequence) and the inner auditory complexity of the sequence.

The inner auditory complexity measure is a variation of the MDL complexity, in the following manner. Here the auditory signal changes with time, and the inner blocks of the sequence are the digits read and those are transients. Thus, there is a single “scanning” method: forward in time. The sequences have different block numbers, ranging from 1 to 6. The compression of a sequence is correlated to the repeating groups of auditory blocks (digits) in the sequence and to its length.

The PDL algorithm reduces simply to:

For each sequence length N, N=2:6,

Compress universally each of the 9^{N} words Wⱼ(N)

Calculate nⱼ(N), j=1:9^{N}, the number of bits required to describe every word.

Cⱼ=nⱼ+log N

The complexity of sequences presented is governed by the subject's performance in the previous sequence trial. Until the first failure, each sequence of length N (N>1) has a complexity index “1” (all digits different, no inner order). If a subject fails in repeating a sequence, another sequence of the same length is read, with lower complexity. This procedure is repeated until a limit of three failures in a row or a success of the subject in repeating the sequence. Upon three failures for a sequence of length N, the test is aborted. Upon a success, the complexity is increased by first increasing the sequence's complexity index to “1”. Success in the increased complexity trial results in incrementing the length of the sequence and repeating the procedure. Failure in the increased complexity trial results in aborting the test.

The maximum length of a sequence in the test is N=6.

### Recall of a Digits Sequence in Reversed Order

As in test 5, in this test a sequence of digits is read, more digits every time (i.e. a sequence of 2, 3 . . . till 6 digits). This time the subject has to repeat the sequence in reversed order. The instructions are the same—the repeating of the digits is by pressing the appropriate digit keys on the keyboard. A white square designates the end of the sequence reading, and the digits typed by the subject are printed in it. The sequence presentation is adaptively determined by the PDL complexity measures, similar to test 5.

### Strike out the Plus Symbols

In this part of the test the subject has to interact with the computer mouse. The instruction and trial period walk the subject concisely and efficiently through the use of the mouse (moving and clicking). Here, the interactive adaptation of the instructions to the subject's acquaintance with the computer media is highly important. The mouse pointer on the screen is drawn as a small hand and subjects who are not familiar with this media are taught to connect the movment of the hand on the screen with their own hand moving the mouse. The length of the instructions is determined by evaluating the mouse trajectories (see test analysis section). The subject is primarily instructed to put his/her hand on the mouse deposited on the table beside the keyboard and to move the mouse left and right (“to see if the mouse is functioning properly”).

In the second trial phase, a square button appears in the middle of the screen, in which a Plus symbol is printed. The subject is instructed to move the small hand, placed in the bottom-left most part of the screen, into the white square and then to press the left mouse button. Upon pressing the button when the mouse is inside the square, the plus symbol disappears.

If motor problems are encountered from the mouse trajectory to the square (which should be close to a straight line, see analysis) this motion is interactively practiced with the subject. If problems are encountered in the clicking action, the mouse is first locked in its position when inside the square and the button pressing is interactively practiced with the subject.

The next phase of the trial simulates a miniature of the actual test: A row with 6 squares is presented in which only two of the square contain a plus while the others contain different symbols, similar to the ones presented in the first test (“spot the plus symbol”). The subject is instructed to move the mouse into every square that contains a plus, and to strike out the symbol by clicking on it. Each of the 3 stages in the training described above is repeated/rephrased and reviewed several times, and a limit number of unsuccessful trials is defined for each stage (3, 3, and 4 respectively). When this limit is reached, the test terminates and the subject thanked and moves on to the last part.

When the trial successfully finished, the test itself presents a matrix of 6 rows with 8 squares in each, that contains randomly places symbols, some of them are pluses. The subject is instructed to strike out the pluses, in a similar manner as the trial period. Like in test 4 (DSST), in every row, two adjacent symbols are set to be pluses. The performance pattern of striking out a pair of such adjacent symbols was found to be very significant.

### Shape Recall After Elapsed Time

In this part of the test the subject is presented with patterns like the ones presented in the “recall a pattern” test and then with digit-symbol pairs like the ones in the template of the DSST. The patterns appear one after the other, for 5 sec each. The subject is instructed to press on any key on the keyboard, when he/she recalls this pattern was among that ones that had to be recalled in test 3. Three patterns are presented, one of which was not present in the previous tests and two from the patterns that had to be recalled in test 3. Then three digit-symbol pairs appear one after the other, for 5 sec each. The subject is instructed to press on any key on the keyboard, when he/she recalls this pattern was among the ones that appeared in the template os the DSST. One of the pairs is randomly chosen from the ones in the DSST template and the two others are different symbols paired with the digits.

### II. Detailed Description of the Tests Analysis

The performance of the users in the tests was evaluated by both the ratio of correct responses to the tasks prescribed and the reaction time (RT).

### Pre-Processing

### Key Press Reaction Time Regulation Analysis

RTs were the latencies from the presentation of each stimulus until an acceptable key was pressed by the subject in response to the stimulus, even if the response was incorrect. Of the two performance measures, the RT was more likely to be affected by computer skill. Therefore, we constructed an adjustment technique to overcome these variations.

Three adjustment factors were introduced. One adjustment for baseline reaction capabilities of the subject used the average RT in the first test (Spot the Plus Symbols), which was subtracted from each RT in subsequent tests of the same subject. The subsequent tests demanded only a slightly more difficult computer interaction (choosing a digit key instead of just hitting any key), while increasing substantially the cognitive difficulty level. The resulting values were denoted corrected reaction time (CRT). The second adjustment factor, which was applied to all four tests, was the training period length of each subject. Preliminary observations suggested that longer RT values corresponded with longer training periods. The length of each training period was the latency from the introduction of the first training stimulus till the subject's response to the last stimulus in the training period. This training length (TL) was recorded for each subject for every test, and used to scale each CRT in that test. The resulting unit-less values were denoted normalized corrected reaction time (NCRT), where NCRT=CRT/TL. Since CRT values were of the order of seconds and TL-of minutes, the NCRT values were of the order of 10⁻².

A third adjustment factor attended the variability in performance for different complexity in tasks within a test. In the second and third tests (Identify the Odd Pattern and Recall a Pattern), a complexity measure, R_{PC}, was calculated for every task. The Spot the Plus symbols test and DSST employed simple symbols and therefore the R_{PC} of all tasks was set to “1”. All NCRT values were multiplied by the R_{PC} corresponding to the task presented. The R_{PC} values thus serve as weights to the NCRT and enables comparison of NCRT for the multiple tasks in each test.

### II. Key presses Feature Extraction and Test Scores

The test scores included two parts: one represented the percentage of correct responses and the other quantified the performance based on NCRT features.

Correct key presses in response to each stimulus presented were logged and were divided by the number of stimuli presented during a test, to yield the feature f0. In the first test (Spot the Plus Symbols) f0 logged key presses in response to a “Plus” symbol. The second, third and fourth tests logged all correct digit key presses.

RT values were logged for each stimulus presented and NCRT were calculated. Three features were extracted from the NCRT data. The first of those features, f1, was the average NCRT in each test (f1=<NCRT>). The second feature was the change in time of NCRT in each test. Linear regression of each NCRT sequence yielded a slope, termed f2 (f2=ΔNCRT). The third feature applied only to the DSST. In every screen of the DSST, there were two adjacent symbols in the filling table, which were set to be identical. For each such pair, the ratio between the NCRT of the second symbol and the NCRT of the first symbol was derived and termed f3. Preliminary results for normal controls showed faster RT to the second stimulus in the pair, presumably since they did not have to do the matching all over again.

The performance based on each of these features, fᵢ, i=1, 2, 3, was calculated as a z score [1]: the fi value position within the distribution of fi values for this test across the control population, in standard deviation units:

$${z}_{i} = \frac{{f}_{i} - 〈 {f}_{i} 〉}{SD}$$

where SD is the standard deviation of the distribution and <fᵢ> is the feature average value across the control population.

The scores of the tests were a weighted sum of the f0 score and the zi scores of NCRT features:

The scores for the three first tests were:

100·(f₀−z₁z₁·w₂z₂)

where w₁=w₂=0.1

And for the DSST:

100·(f₀−w₁z₁−w₂z₂−w₃z₃)

where w₁=0.1 and w₂=w₃=0.05

### Mouse Handling Features and Analysis

In the seventh part of the test, the input device is changed and mouse clicking replace the key presses.

The baseline for evaluating mouse movements is determined in two steps of the training period:

A small hand icon appears on the screen and the subject is primarily instructed to put his/her hand on the mouse deposited on the table beside the keyboard and to move the mouse left and right. The mouse trajectories are recorded and their direction and speed of movement are calculated. The features extracted are the speed (v1) and the variance of the movement from a straight, horizontal line, calculated by matching the trajectory linear segments, yielding an average match v2.

In a second training step, the hand icon is locked in the lower left corner on the screen, until, upon instruction, the subject moves the mouse “so that the hand reaches the button in the middle of the screen”. The mouse trajectory is matched to a straight diagonal line, yielding new feature v1 and v2. The mouse stopping manner (stable or the amount of shifting) and location (inside or the amount outside the button) are evaluated into the features v3 and v4, respectively.

The subject then clicks on the mouse and the click latency (v5) and persistency (v6) is recorded.

The average values of v1 to v6 across all movements in the training serve as a baseline values to the same features extracted in the test, where similar actions are performed, coupled to button selection task.

The test performance parameters are the set of values v1 to v6 divided by the values of the baseline average features:

$${\{ {f}_{i} \}}_{i = 1}^{6} = \frac{{v}_{i} ( test )}{{v}_{i} ( baseline )}$$

Z values are calculated for all fᵢ and the score of the Strike Out the plus Symbols is determined by f₀, the mistakes ratio and the z values as follows:

$$100 \cdot ( {f}_{0} - {\sum }_{i = 0}^{6 } {w}_{i} {z}_{i} )$$

where, wᵢ=0.04 for all i.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method to be performed on a computer for neuropsychological evaluation of a person, the method comprising:
calculating complexities of a plurality of stimuli by a pattern description length algorithm for a pattern of each said stimulus;
presenting stimuli to said user in reaction to which said user has to perform a cognitive test, said stimuli being presented in order of complexity, and
adjusting a complexity level for a next cognitive test based on said user's response.

2. A method according to claim 1 wherein said pattern description length algorithm for a stimulus j is:
PDL₍ⱼ₎minᵢ[nⱼ(i)+log pᵢ(i)]
wherein n is a word description length, n=L−S where L is a Lempel-Ziv code and S is a description-facilitating sequence; wherein i is a tracing through a pattern of said stimulus; wherein nⱼ(i) is the number of bits required to describe a word created from pattern j using tracing i and wherein pⱼ(i) is the a-priori probability for compression using each tracing i.

3. A method according to claim 1 wherein calculating said complexity includes a measure reflecting a ratio between complexities of stimuli.

4. A method according to claim 3, wherein a task complexity ratio for stimuli patterns A and B is R_{PC}=P_{C}(A)*P_{C}(A)/P_{C}(B).

5. A method to be performed on a computer for neuropsychological evaluation of a person, the method comprising:
presenting tests containing stimuli in reaction to which said person has to perform a cognitive task, said tests having multiple tasks of different complexity levels and wherein said complexity levels are associated with said stimuli;
during prior training of the person on a test and during said test:
defining an expected response to said stimuli,
measuring the response of said person to the stimuli associated with the test,
analyzing said response,
reducing a complexity of stimuli for a next task if said person incorrectly responds to stimuli of said task, and
increasing a complexity of stimuli for a next task if said person correctly responds to stimuli of said task;
calculating said complexity for each next task prior to determining said next task;
changing an expected response time of a particular stimuli as a function of said person's responses;
storing results from said tests; and
evaluating performance of said person in reaction to stimuli of said tests.

6. The method according to claim 5 and also comprising:
determining a score S for each person, wherein
$$S = {\sum }_{i}^{ } {w}_{i} {f}_{i} < T$$
and wherein T is a threshold defining a disorder, fᵢ are a set of features from the received responses in a test and wᵢ are per feature weights based on population statistics.

7. The method according to claim 5 and also comprising:
calculating complexities of a plurality of stimuli by a pattern description length algorithm for a pattern of each said stimulus;
presenting stimuli to said user in reaction to which said user has to perform a cognitive test, said stimuli being presented in order of complexity, and
adjusting a complexity level for a next cognitive test based on said user's response.
